(19) United States
(12) Patent Application Publication
(10) Pub. No.: US 2025/0000785 A1
(43) Pub. Date: Jan. 2, 2025

(54) COSMETIC COMPOSITION COMPRISING CRANBERRY EXTRACT AND CRANBERRY SEED OIL

(71) Applicant: Givaudan SA, VERNIER (CH)

(72) Inventors: Morgane DE TOLLENAERE, Pomacle (FR); Amandine SCANDOLERA, Pomacle (FR); Romain REYNAUD, Toulouse (FR)

(21) Appl. No.: 18/695,696

(22) PCT Filed: Sep. 29, 2022
(86) PCT No.: PCT/EP2022/077096

§ 371 Date: Mar. 26, 2024

(30) Foreign Application Priority Data
Oct. 1, 2021 (GB) 2114116.3

(51) Int. Cl. A61K 8/9789; A61K 8/19; A61K 8/24; A61K 36/45; A61K 47/02; A61P 29/00; A61Q 19/00

(52) U.S. Cl. CPC A61K 8/9789; A61K 8/19; A61K 8/24; A61K 36/45; A61K 47/02; A61P 29/00; A61Q 19/00; A61K 2800/75

(57) ABSTRACT

The present invention provides a cosmetic composition for topical use, comprising cranberry extract in combination with cranberry seed oil.

The present invention relates to a cosmetic composition for topical use, comprising a combination of cranberry extract and cranberry seed oil. In particular, the invention is concerned with a cosmetic composition for topical use, comprising a combination of cranberry extract and cranberry seed oil, wherein the cranberry extract and the cranberry seed oil are in a ratio between about 15:1 to about 1:0.5. The invention also relates to a cosmetic product comprising the cosmetic composition comprising cranberry extract in combination with cranberry seed oil as defined herein, as well as to a method for alleviating the symptoms of inflammation of the human skin or the human scalp, comprising topically applying to the skin or the scalp the cosmetic composition or the cosmetic product as defined herein. The invention is also concerned with the use of the cosmetic composition or the cosmetic product as defined herein in alleviating the symptoms of inflammation of the human skin or the human scalp.

### BACKGROUND

Cranberries (Vaccinium macrocarpon) contain large quantities of antioxidants, vitamins, minerals and phenolic compounds, which prompted their increasing use in various food products and nutraceutical supplements. Extracts from cranberries have also been used in a number of cosmetics, due to their anti-glycation, anti-microbial, antioxidant and anti-aging properties.

The seeds, separated from the fruit, are the source of cranberry seed oil. Cranberry seed oil provides a distinct combination of omega-3, omega-6 and omega-9 fatty acids, as well as tocopherols, tocotrienols and a high concentration of antioxidants. The oil has been used in a number of cosmetics, due to its moisturizing, nourishing, anti-aging, antioxidant, anti-microbial, UV protecting, anti-irritant, and skin-lightening properties.

Skin inflammation, in an interdependent relationship with oxidative stress, can manifest itself in a number of ways, such as redness, breakouts, acne, blemishes, rosacea, dark spots, hyperpigmentation, eczema, itching, or swelling, among others. Inflammation of the scalp, although harder to identify, can be observed as flakiness, swelling or redness of the scalp.

In order to counteract the unpleasant feel and appearance of skin and scalp inflammation, commercial soothing compositions have been marketed. Of these compositions, those containing natural extracts have become more popular in recent years. Most natural soothing compositions contain extracts from arnica, aloe vera, calendula, chamomile (containing bisabolol), coconut oil, green tea, oatmeal, shea butter and tea tree oil, for example.

Although not as widely used as the above-mentioned ingredients, cranberry extracts have also been shown to have skin and scalp soothing properties. It is believed that the high concentration of cranberry polyphenols is responsible for such effects.

There is still a need to provide compositions containing natural and/or upcycled ingredients that have a soothing effect on the human skin or scalp (i.e. that can reduce or alleviate the symptoms of skin or scalp inflammation), in particular compositions based on cranberry components.

### SUMMARY OF THE INVENTION

In a first aspect, the invention provides a cosmetic composition for topical use, comprising a cranberry extract in combination with cranberry seed oil.

In a further aspect, a cosmetic product comprising a cosmetic composition for topical use, comprising cranberry extract in combination with cranberry seed oil as defined herein and a cosmetically acceptable base is provided.

The invention further provides a method, in particular a cosmetic method, for alleviating or removing the symptoms of inflammation of the human skin or the human scalp, comprising applying to the skin or the scalp a cosmetic composition or a cosmetic product as defined herein.

In yet another aspect, use of a cosmetic composition or a cosmetic product as defined herein in alleviating or removing the symptoms of inflammation of the human skin or the human scalp is provided.

### BRIEF DESCRIPTION OF THE DRAWINGS

FIG. 1 shows a non-parametric statistical analysis using a Mann Whitney test for TNF-α quantification in samples of culture cells treated with cranberry extract, cranberry seed oil and combinations of cranberry extract and cranberry seed oil, respectively, compared with a sample treated with a reference (dexamethasone).

### DETAILED DESCRIPTION

Preferred and/or optional features of the invention will now be set out. Any aspect of the invention may be combined with any other aspect of the invention unless the context demands otherwise. Any of the preferred or optional features of any aspect may be combined, singly or in combination, with any aspect of the invention, as well as with any other preferred or optional features, unless the context demands otherwise.

### Cosmetic Composition

It has surprisingly been found that a combination of cranberry extract and cranberry seed oil showed synergistic anti-inflammatory activity by significantly reducing the release of cytokine TNF-α by keratinocytes stressed with phorbol 12-myristate 13-acetate (PMA). In particular, it was found that the TNF-α release was not only significantly reduced when a combination of cranberry extract and cranberry seed oil was employed compared to when each of the cranberry extract and the cranberry seed oil were employed independently, but also it was much lower than the TNF-α release expected by adding the effects of the two independent components.

The invention, therefore, provides a cosmetic composition for topical use, comprising a combination of cranberry extract and cranberry seed oil.

The cranberries from which both the cranberry extract and cranberry seed oil are obtained May be sourced from the United States, for example.

### Cranberry Extract

The cranberry extract is obtained from Vaccinium macrocarpon berries and, preferably, from the juice obtained by crushing them.

For example, the cranberry extract can be obtained by a process of extraction from cranberry fruit. After harvesting, drying and grinding, the cranberries may be contacted with a solvent that has an affinity for the desired components to be extracted. Subsequently, a filtration step may separate the solvent in which the desired components are dissolved from the cranberry solid matrix, providing a native extract. The solvent may then be removed, for example evaporated, in order to concentrate the filtrate. The resulting native extract may be subjected to further processing by separation, purification and/or further formulation. Suitable methods of extraction include ultrasound, microwave-assisted or Soxhlet extraction.

The solvents that may be used in the extraction process may be hydrophilic or lipophilic. Depending on the solvent used, the cranberry extract may be called a “hydrophilic extract” or a “lipophilic extract”.

In an embodiment, the cranberry extract is a hydrophilic extract.

Examples of suitable hydrophilic solvents that may be used in the extraction process include, but are not limited to, water; alcohols, such as methanol, ethanol, propanols (normal or iso-), butanols (normal, iso- or tert-); ketones, such as acetone; nitriles, such as acetonitrile; or mixtures thereof. Optionally, acids, such as hydrochloric or sulfuric acid, or organic acids such as trichloroacetic, trifluoroacetic, formic, citric or acetic acid, or mixtures thereof may be used to facilitate the extraction.

In one embodiment, the cranberry extract is an upcycled product obtained after partial extraction of the polyphenols from cranberry juice.

In an embodiment, the cranberry extract comprises organic acids, such as phenolic acids, especially those derived from hydroxybenzoic and hydroxycinnamic acid, quinic, malic, shikimic, and citric acid.

In an embodiment, the solvent employed to extract the desired components may be a water-ethanol mixture.

In an embodiment, the cranberry extract may contain between about 7% to about 30% w/w of organic acids. For instance, the cranberry extract may contain about 7%, about 18% or about 30% organic acids by weight of the extract.

It has been shown that polyphenolic plant sources possess antioxidant activity and could potentially prevent and control oxidative stress and its effects (Silva Caldas, A. P., International Journal of Food Properties, 2018, 21, 582-592, the disclosure of which is herein incorporated by reference). Therefore, it is advantageous to use a cranberry extract with a higher content of organic acids, such as phenolic acids.

Therefore, in one embodiment, the cranberry extract contains more than about 30% organic acids by weight of the extract.

Optionally, the cranberry extract may be further mixed with an additive. Additives are substances added to natural extracts to maintain or improve their stability, texture, or appearance.

For example, the additive may be selected from the group consisting of sodium benzoate, potassium sorbate, magnesium hydroxide, tricalcium phosphate, glycerine and mixtures thereof. Their role is to stabilize the pH of the cranberry extract.

In one embodiment, the additive may be magnesium hydroxide. Optionally, the cranberry extract may contain a further additive, such as tricalcium phosphate.

In one embodiment, the cranberry extract may contain about 10% w/w magnesium hydroxide.

In one embodiment, the cranberry extract may contain less than about 1% w/w of tricalcium phosphate.

In one embodiment, the cranberry extract may contain about 10% w/w magnesium hydroxide and less than about 1% w/w of tricalcium phosphate.

The cranberry extract may be in any suitable form, for example in a liquid or powder form.

In an embodiment, the cranberry extract may undergo further processing, such as drying. Optionally, an additive may be added to the cranberry extract prior to the drying step.

For example, the cranberry extract may be obtained by a process as described herein. The cranberry fruit may be ground, extracted with a mixture of water and ethanol and then filtered. The filter cake may be discarded and the solvent may be removed from the filtrate, for example by concentration in order to obtain a native extract of cranberry. The native extract may be further processed by removing a polyphenols-rich fraction. The fraction left after removal of the polyphenol-rich fraction may be mixed with magnesium hydroxide and/or tricalcium phosphate, the mixture may be dried, e.g. by spray drying, and, optionally, it may further undergo sieving.

### Cranberry Seed Oil

The cranberry seed oil possesses a distinct combination of omega-3 fatty acids (about 26% to 34% w/w), omega-6 (about 32% to 42% w/w) and omega-9 (about 18% to 30% w/w), as well as tocopherols, tocotrienols and antioxidants.

A number of extraction techniques, such as solvent, ultrasonic-assisted, super-critical fluid, Soxhlet extraction or mechanical pressing, may be used to extract the cranberry seed oil.

Because some compounds found in cranberry seed oil may be destroyed or removed during steam stripping or solvent extraction processes, preferably the cranberry seed oil is obtained by cold pressing, resulting in virgin (i.e. an oil produced by only physical or mechanical means) cranberry seed oil. In one embodiment, therefore, the cranberry seed oil may be a virgin cranberry seed oil.

For example, the cranberry seed oil may be obtained by a process as described herein. The seeds removed from the fruit may be cold pressed and filtered. The filtration cake may be discarded and the filtrate (i.e. the virgin cranberry seed oil) may be used as is.

### Cranberry Extract and Cranberry Seed Oil

As has been discussed above, both cranberry extracts and cranberry seed oils have been used in cosmetic products due to their beneficial properties when applied to the skin. However, no study evaluating the anti-inflammatory effect of cranberry extracts or cranberry seed oils has been undertaken to date.

It was surprisingly and unexpectedly observed that, by treating keratinocytes stressed with phorbol 12-myristate 13-acetate (PMA) with a combination of cranberry extracts and cranberry seed oils, the release of cytokine TNF-α was significantly reduced compared to treatment with only cranberry extract or cranberry seed oil independently. The effect of the combination of cranberry extracts and cranberry seed oils on the release of cytokine TNF-α was much higher than the effect expected by simply adding up the effects of the two independent components. Thus, a synergistic effect of the combination of cranberry extracts and cranberry seed oils can be clearly observed (see, e.g., example 3 below).

In one embodiment, the ratio between the cranberry extract and cranberry seed oil is between about 15:1 to about 1:0.2. Preferably, the ratio between the cranberry extract and cranberry seed oil is between about 12:1 to about 1:0.5. For example, the ratio between the cranberry extract and cranberry seed oil may be about 12:1, 11:1, 10:1, 9:1, 8:1, 7:1, 6:1, 5:1, 4:1, 3:1, 2:1, 1:1, 1:0.9, 1:0.8, 1:0.7, 1:0.6 or 1:0.5, in particular about 10:1 or about 1:1.

### Cosmetic Product

Another aspect of the present invention provides a cosmetic product comprising a cosmetic composition comprising cranberry extract in combination with cranberry seed oil as defined hereinbefore and a cosmetic base.

The cosmetic base may comprise any cosmetically acceptable excipient. A cosmetically acceptable excipient can be any excipient commonly used in the preparation of cosmetic preparations for use on the human skin or scalp. Suitable excipients include, but are not limited to, ingredients that can influence organoleptic properties and penetration of the skin, as well as the amount of time the active ingredient(s) remain active on the skin. More specifically, they include liquids, such as water, oils or surfactants, including those of petroleum, animal, plant or synthetic origin, such as and not restricted to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, digitonin, and the like, or combinations thereof.

The formulation for topical application to the skin may take any physical form. For instance, the cosmetic product may be in the form of a liposome, mixed liposomes, oleosomes, niosomes, ethosomes, milliparticles, microparticles, nanoparticles and solid-lipid nanoparticles, vesicles, micelles, mixed micelles of surfactants, surfactant-phospholipid mixed micelles, millispheres, microspheres and nanospheres, lipospheres, millicapsules, microcapsules and nanocapsules, as well as microemulsions and nanoemulsions, which can be added to achieve a greater penetration of the cosmetic composition as defined herein.

The cosmetic product may be produced in any solid, liquid, or semi-solid form useful for application to the skin topically or by transdermal application. Thus, these preparations of topical or transdermal application include, but are not restricted to, creams, multiple emulsions, such as and not restricted to, oil and/or silicone in water emulsions, water-in-oil and/or silicone emulsions, water/oil/water or water/silicone/water type emulsions, and oil/water/oil or silicone/water/silicone type emulsions, micro-emulsions, emulsions and/or solutions, liquid crystals, anhydrous compositions, aqueous dispersions, oils, milks, balsams, foams, aqueous or oily lotions, aqueous or oily gels, cream, hydro-alcoholic solutions, hydro-glycolic solutions, hydrogels, liniments, sera, soaps, face masks, serums, polysaccharide films, ointments, mousses, pomades, pastes, powders, bars, pencils and sprays or aerosols (sprays), including leave-on and rinse-off formulations.

The cosmetic product of the present invention may also be any product typically used for hair care, including but not limited to a shampoo, conditioner, spray, treatment, mask, strengthener, pre-shampoo, lotion, serum, cream, foam, mousse, and gel. Particularly preferably, the cosmetic product is a shampoo, anti-frizz hair spray, beauty hair mask, hair shininess serum, hair conditioner, hair strengthener pre-shampoo, or hair protection lotion. Both leave-on and rinse-off product types are suitable.

The cosmetic product according to the present invention may further comprise one or more materials selected from the group consisting of carriers, solvents, surfactants, thickeners, styling polymers, anti-dandruff actives, antimicrobial materials, skin and scalp actives, vitamins, salts, buffers, hair growth agents, conditioning materials, hair-fixative polymers, fragrances, colorings/colorants, dyes, pigments, opacifiers, pearlescent aids, oils, waxes, preservatives, sensates, sunscreens, medicinal agents, antifoaming agents, antioxidants, binders, biological additives, buffering agents, bulking agents, chelating agents, chemical additives, film formers or materials, pH adjusters, propellants, oxidizing agents, and reducing agents.

The cosmetic product of the present invention may be produced in any form typically used for skin or scalp care, including but not limited to a cream, a lotion, a spray, a gel, a foam a stick, a shampoo, a conditioner or a mousse, preferably a cream or a gel.

The cosmetic compositions and products of the present invention can additionally further comprise any suitable optional ingredients as desired. Such optional ingredients should be physically and chemically compatible with the essential components of the cosmetic composition or cosmetic product, and should not otherwise unduly impair stability, aesthetics or performance. The CTFA Cosmetic Ingredient Handbook, Tenth Edition (published by the Cosmetic, Toiletry, and Fragrance Association, Inc., Washington D.C.) (2004) describes a wide variety of non-limiting materials that can be added to the compositions and products of the present invention.

The concentration of the cranberry extract in a topical formulation of a cosmetic composition can be up to about 30% w/w. In one embodiment, the cosmetic product comprises about 0.5% to about 30%, optionally about 1% to about 25%, optionally about 5% to about 15% w/w of cranberry extract.

In one embodiment, the cranberry extract may be provided in powder form and may be mixed with a solvent such as water, ethanol/water or glycerine, before incorporation in a cosmetic product.

The concentration of the cranberry seed oil in a topical formulation of a cosmetic product can be up to about 10% w/w. In one embodiment, the cosmetic product comprises about 0.05% to about 10%, optionally about 0.5% to about 5%, optionally about 1% to about 3% w/w of cranberry seed oil.

### Methods of Use

The invention further provides a method for alleviating or removing the symptoms of inflammation of the human skin or the human scalp, comprising topically applying to the skin or the scalp a composition comprising cranberry extract in combination with cranberry seed oil or a cosmetic product comprising a composition comprising cranberry extract in combination with cranberry seed oil as defined hereinbefore.

In particular, the invention provides a cosmetic method for alleviating or removing the symptoms of inflammation of the human skin or the human scalp, i.e. a non-therapeutic method.

In one embodiment, the inflammation of the human skin or the human scalp may be caused by an infection, by an allergic reaction or by exposure to UV radiation.

In one embodiment, the inflammation of the human skin may be selected from the group consisting of sensitive skin, irritated skin, atopic dermatitis, rosacea, eczema and psoriasis.

In one embodiment, the inflammation of the human scalp may be selected from the group consisting of dermatitis, such as irritated skin due to dryness, itchy scalp, seborrhoeic dermatitis and contact dermatitis, scalp folliculitis and psoriasis of the scalp.

Non-limiting examples of symptoms of inflammation include redness, breakouts, acne, blemishes, rosacea, dark spots, hyperpigmentation, eczema, itching, or swelling of the skin and flakiness, swelling or redness of the scalp.

In yet another aspect, use of a composition comprising cranberry extract in combination with cranberry seed oil or a cosmetic product comprising a composition comprising cranberry extract in combination with cranberry seed oil as defined hereinbefore in alleviating or removing the symptoms of inflammation of the human skin or the human scalp is provided.

In particular, the invention provides a cosmetic use for alleviating or removing the symptoms of inflammation of the human skin or the human scalp, i.e. a non-therapeutic use.

The present invention is further illustrated by means of the following non-limiting examples:

### Example 1: Process of Production of a Cranberry Extract

The cranberry fruit may be ground, extracted with a mixture of water and ethanol and filtered. The filter cake may be discarded and the filtrate may undergo concentration/desolventation in order to obtain a native extract of cranberry. The resulting native extract is further subjected to separation, purification and further formulation by mixing with magnesium hydroxide and tricalcium phosphate. The mixture may be dried by spray drying and, optionally, it may further undergo sieving.

For example, 134 kg of cranberries were ground and extracted with 80 kg of water. The liquid was separated from the solid mass, concentrated and subjected to further processing, comprising the step of mixing with magnesium hydroxide (8 kg) and tricalcium phosphate (1 Kg). 92 kg of cranberry extract with 30% w/w organic acids was obtained.

### Example 2: Process of Production of a Cranberry Seed Oil

The seeds removed from the fruit may be cold pressed and filtered. The filtration cake may be discarded and the filtrate (i.e. the virgin cranberry seed oil) may be used as is. For example, 1.667 kg of cranberries were squeezed and sliced to remove the seeds. The seeds (about 17 g) were collected, dried and cold pressed to obtain about 1 g of cranberry seed oil.

### Example 3: Anti-Inflammatory Effect of a Cranberry Extract, a Cranberry Seed Oil and Combinations of a Cranberry Extract and a Cranberry Seed Oil

The effect of a cranberry extract, a cranberry seed oil and combinations of a cranberry extract and a cranberry seed oil on the anti-inflammatory activity of keratinocytes stressed with PMA was evaluated by comparing the decrease of the cytokine TNF-α release of Normal Human Epidermal Keratinocytes (NHEKs) treated with the cranberry products and stressed with PMA (Phorbol 12-myristate 13-acetate) with the TNF-α release of untreated NHEKs stressed with PMA.

### 3.1. Cell Culture and Treatment

The cell culture was done on primary cells isolated from fresh biopsies. NHEKs were seeded in a type I collagen pre-coated 24 wells-plate at 30 000 cells per well in triplicate.

The cells were incubated 24 hours in complete medium (Epilife medium supplemented with HKGS, Gibco) and 1% of antibiotics (Sigma-Aldrich) at 37° C. with 5% CO₂.

At the end of the incubation, the cells were rinsed twice with phosphate buffered saline (PBS, Gibco) and pre-incubated with active components for 24 hours in Epilife complete medium (Epilife+Human keratinocyte growth supplements (HKGS) supplements, Gibco) without Hydrocortisone and 1% of antibiotics at 37° C. with 5% CO₂.

After 24 hours of pre-incubation, the cells were stressed with PMA (Phorbol 12-myristate 13-acetate, Sigma) at 1 ng/ml and incubated for 24 hours at 37° C. with 5% CO₂.

The compounds used for the comparison are as shown in Table 1. Non-treated cells were used as a blank. Cells treated with a known anti-inflammatory (dexamethasone) were used as a reference.

TABLE 1
Compounds and cranberry products tested for
their anti-inflammatory effect on NHEKs

| | Active components | Concentration |
|---|---|---|
| 1 | None (blank) | |
| 2 | Dexamethasone | 1 μM |
| 3 | Cranberry extract | 0.5 mg/mL (w/v) |
| 4 | Cranberry seed oil | 0.05% (v/v) |
| 5 | Cranberry extract + Cranberry | 0.5 mg/mL (w/v) + 0.05% (v/v) |
| | seed oil (ratio 1:1) | |
| 6 | Cranberry seed oil | 0.005% (v/v) |
| 7 | Cranberry extract + Cranberry | 0.5 mg/mL (w/v) + 0.005% (v/v) |
| | seed oil (ratio 10:1) | |

Skin cells untreated and cultivated with medium were used as negative control.

At the end of the culture, the cell media were collected and centrifuged at 2000 g for 10 minutes at 4° C. to eliminate dead cells. The media were stored at −20° C.

A MTT assay was performed to evaluate treatments toxicity and for the normalisation of TNF-α quantification.

The assay was done in quadruplicate.

### 3.2. MTT Assay

MTT solution (Sigma) diluted at 1 mg/mL in basal medium was added to each well and incubated for 3 hours at 37° C., 5% CO₂. Then, the medium was removed and 300 μL of dimethyl sulfoxide (DMSO, Sigma) was added into each well to dissolve the formazan crystals. Homogenization was done under orbital agitation for few minutes. The optical density was measured at a wavelength of 560 nm.

### 3.3. TNF-α Quantification

The TNF-α quantification was realized with the Human TNF-alpha Quantikine ELISA kit (DTA00D, R&D Systems).

The samples and the standard range were incubated for 2 hours in 96-wells plate pre-coated with a monoclonal antibody specific for human TNF-α in presence of the Assay Diluent RD1F. After four washes with wash buffer provided by the supplier, a polyclonal antibody specific for human TNF-α conjugated to horseradish peroxidase enzyme (HRP) was incubated for 2 hours under orbital agitation. Following 4 washes, a substrate solution was added to the wells for 30 minutes in the dark. The catalysis of this substrate by HRP generates blue colour in proportion to the amount of TNF-α bound in the initial step. The colour development was stopped by a Stop solution and changed to yellow colour. The optical density was measured at 450 nm and 540 nm with a microplate reader (TECAN SPARK® 10M).

The Four Parameter Logistic Curve analysis was performed via the Myassays website (http://myassays.com) after subtraction of optical density at 450 nm by optical density at 540 nm.

The quantification was carried out in duplicate.

The quantification was normalized with the optical density measured via the MTT assay.

### 3.4. Statistical Analysis

A Shapiro-Wilk normality test revealed that the samples did not follow the Gaussian Law. Consequently, a non-parametric statistical analysis was performed with Kruskal-Wallis ANOVA followed by Mann Whitney U test. A result was considered significant with p<0.1 (marked with #), p<0.05 (marked with *), p<0.01 (marked with **) and p<0.001 (marked with ***).

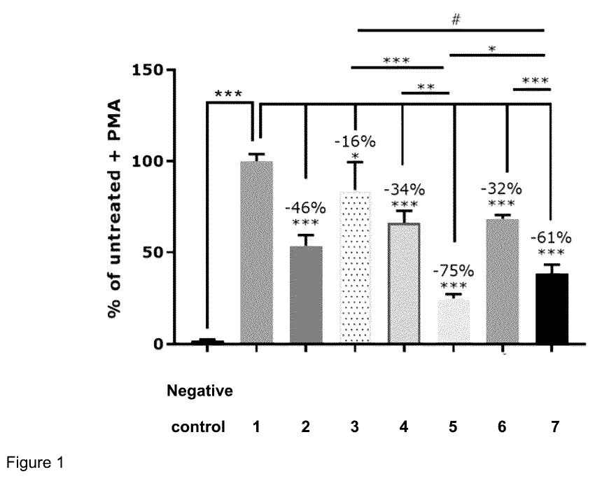

FIG. 1 shows the results of the non-parametric statistical analysis using the Mann Whitney test for samples 1 to 7 in Table 1.

The quantification demonstrated that, after 24 hours of the PMA stress (entry 1 in Table 1), the keratinocytes significantly released cytokine TNF-α. The pre-treatment with dexamethasone (1 μM, entry 2) significantly reduced the release of the cytokine by 46% ***. These results confirmed the responsiveness of the model.

In the same conditions, the cranberry extract at 0.5 mg/mL (entry 3), the cranberry seed oil at 0.05% v/v (entry 4) and 0.005% v/v (entry 5) significantly reduced the TNF-α release by 16% *, 34% *** and 32% ***, respectively, compared to the sample of entry 1.

In the presence of a combination of cranberry extract at 0.5 mg/ml and cranberry seed oil at 0.05% v/v (i.e. a ratio of 1:1, entry 4), the decrease of TNF-α release was significantly higher (75% ***). The reduction was significantly better than what would have been expected by adding up the reduction due to each component individually.

In the presence of a combination of cranberry extract at 0.5 mg/ml and cranberry seed oil at 0.005% v/v (i.e. a ratio of 10:1, entry 6), the decrease of TNF-α release of 61% *** was significant (p<0.001). The decrease was higher than what would have been expected by simply adding up the reduction due to each component individually.

Therefore, it has been shown that a combination of cranberry extract and cranberry seed oil has synergistic anti-inflammatory capability compared to each of the cranberry extract and cranberry seed oil independently.

1. A cosmetic composition for topical use, comprising cranberry extract in combination with cranberry seed oil.

2. The cosmetic composition according to claim 1, wherein the cranberry extract is a hydrophilic extract.

3. The cosmetic composition according to claim 1, wherein the cranberry extract contains more than about 30% w/w organic acids.

4. The cosmetic composition according to claim 1, wherein the cranberry extract further comprises an additive.

5. The cosmetic composition according to claim 4, wherein the additive comprises magnesium hydroxide and/or tricalcium phosphate.

6. The cosmetic composition according to claim 5, comprising about 5% to about 10% w/w magnesium hydroxide and/or less than about 1% w/w of tricalcium phosphate.

7. The cosmetic composition according to claim 1, wherein the cranberry seed oil is a virgin cranberry seed oil.

8. The cosmetic composition according to claim 1, wherein the cranberry extract and the cranberry seed oil are in a ratio of between about 15:1 to about 1:0.5.

9. A cosmetic product comprising the cosmetic composition according to claim 1, and a cosmetically acceptable base.

10. The cosmetic product according to claim 9, wherein the product is provided as a cream, a lotion, a spray, a gel, a foam, a stick, a shampoo, a conditioner or a mousse.

11. The cosmetic product according to claim 9, wherein the cosmetic product comprises about 0.5% to about 30% w/w of cranberry extract and about 0.05% to about 10% w/w cranberry seed oil.

12. A method for alleviating or removing the symptoms of inflammation of the human skin or the human scalp, comprising topically applying to the skin or the scalp the cosmetic composition according to claim 1.

13. The method of claim 12, wherein the inflammation of the human skin or the human scalp is caused by an infection, by an allergic reaction or by exposure to UV radiation.

14. The method of claim 12, wherein the inflammation of the human skin is selected from the group consisting of sensitive skin, irritated skin, atopic dermatitis, rosacea, eczema and psoriasis.

15. The method of claim 12, wherein the inflammation of the human scalp is selected from the group consisting of dermatitis, such as irritated skin due to dryness, itchy scalp, seborrhoeic dermatitis and contact dermatitis, scalp folliculitis and psoriasis of the scalp.

16. (canceled)

17. The cosmetic composition according to claim 2, wherein the cranberry extract contains more than about 30% w/w organic acids.

18. The cosmetic composition according to claim 17, wherein the cranberry extract further comprises an additive.

19. The cosmetic composition according to claim 18, wherein the additive comprises magnesium hydroxide and/or tricalcium phosphate.

20. The cosmetic composition according to claim 19, comprising about 5% to about 10% w/w magnesium hydroxide and/or less than about 1% w/w of tricalcium phosphate.
